(19) United States
(12) Patent Application Publication
(10) Pub. No.: US 2018/0243345 A1
(43) Pub. Date: Aug. 30, 2018

(54) METHOD FOR INCREASING MUSCLE GROWTH USING KRILL EXTRACT

(71) Applicant: AVOCA, INC., Merry Hill, NC (US)

(72) Inventors: David McCray PEELE, Merry Hill, NC (US); Ralf JÄGER, Milwaukee,, WI (US); Martin PURPURA, Austin, TX (US)

(21) Appl. No.: 15/753,128

(22) PCT Filed: Aug. 22, 2016
(86) PCT No.: PCT/US2016/048079

§ 371 Date: Feb. 15, 2018

Related U.S. Application Data
(60) Provisional application No. 62/207,656, filed on Aug. 20, 2015.

(51) Int. Cl. A61K 35/612; A61K 31/685; A61K 31/683

(52) U.S. Cl. CPC A61K 35/612; A61K 31/683; A61K 31/685

(57) ABSTRACT

The present invention relates to methods for conferring mTOR mediated physiological benefits by administering a composition having phospholipids, omega-3 fatty acids, and an antioxidant, or a krill extract to a mammal in need thereof.

This application claims the benefit of U.S. Provisional Application No. 62/207,656, filed Aug. 20, 2015, which is hereby incorporated by reference in its entirety.

### BACKGROUND OF THE INVENTION

The present invention relates to methods of activating the mTOR pathway and effecting processes affected by the mTOR pathway in a mammal, e.g. human.

Increasing or maintaining skeletal muscle mass is an important target for a wide range of populations ranging from athletes (to increase strength and power) to the elderly (to prevent age related muscle loss (sarcopenia) to the immobilized (e.g. to prevent muscle loss during hospitalization (muscle disuse). Skeletal muscle mass is largely dependent upon muscle protein synthesis (MPS), and a protein kinase called the mechanistic target of rapamycin (mTOR) has been widely recognized as a regulator of cellular growth. Specifically, elevations in energy status, amino acids, and growth factors increase MPS through an mTOR-dependent mechanism. Several studies have also shown that signaling by mTOR is required for mechanically-induced increases in MPS and the ultimate hypertrophic response.

The activation of the Akt/mTOR pathway and its downstream targets, like p70S6K, is essentially involved in regulating skeletal muscle fiber size, and that activation of the Akt/mTOR pathway can oppose muscle atrophy induced by disuse.

Besides the skeletal muscular system, mTOR plays a major role in cognitive functioning. The prevailing view on memory formation is that new protein synthesis is required following a learning experience. Several studies have implicated that the mechanistic target of rapamycin (mTOR) is necessary for long-term memory and is involved in memory processing. Prior work suggests that hippocampus-dependent memory undergoes a systems consolidation process such that recent memories are stored in the hippocampus, while older memories are independent of the hippocampus and instead dependent on cortical areas. Activity of mTOR signaling pathway is known to be important for controlling protein translation necessary for both memory consolidation after initial learning and for the reconsolidation of memory after retrieval.

Therefore, there remains a need for improved methods to modulate the mTOR pathway to promote maintain or increase muscle mass, and improve and maintain cognitive function.

### SUMMARY OF THE INVENTION

The invention is a method of activating the mTOR pathway and effecting physiological processes affected by the mTOR pathway. In particular, the method of the invention augments muscle growth and/or strength effected by activation of mTOR pathway in a mammal by administering a composition including phospholipids, omega-3 fatty acids, and an antioxidant, or a hill extract to the mammal before, during, or after physical exercise.

The invention also provides a method of mediating muscle disuse atrophy effected by activation of mTOR pathway in a mammal in need thereof by administering a composition having phospholipids, omega-3 fatty acids, and an antioxidant, or a hill extract to the mammal before, during, or after physical exercise.

Another embodiment of the invention provides a method of mediating age-related muscle loss effected by activation of mTOR pathway in a mammal in need thereof by administering a composition having phospholipids, omega-3 fatty acids, and an antioxidant, or a krill extract to the mammal before, during, or after physical exercise.

In yet another embodiment, the invention provides a method of mediating age-related cognitive decline effected by activation of mTOR pathway in a mammal in need thereof by administering a composition having phospholipids, omega-3 fatty acids, and an antioxidant, or a krill extract to the mammal before, during, or after physical exercise.

In yet another embodiment, the invention provides a method of increasing muscle mass effected by activation of mTOR pathway in a mammal in need thereof by administering a composition having phospholipids, omega-3 fatty acids, and an antioxidant, or a krill extract to the mammal before, during, or after physical exercise.

In yet another embodiment, the invention provides a method of reducing fat mass effected by activation of mTOR pathway in a mammal in need thereof by administering a composition having phospholipids, omega-3 fatty acids, and an antioxidant, or a krill extract to the mammal before, during, or after physical exercise.

As a result of the present invention, muscle related benefits effected by the mTOR pathway can be provided by the administering a composition having phospholipids, omega-3 fatty acids, and an antioxidant, or a krill extract to a mammal in need thereof. Examples of muscle related benefits include augmenting muscle growth and/or strength, mediating muscle disuse atrophy, and mediating age-related muscle loss.

Furthermore, as a result of the present invention, age-related cognitive decline effected by the mTOR pathway can be provided by administering a composition having phospholipids, omega-3 fatty acids, and an antioxidant, or a hill extract to a mammal in need thereof.

### DESCRIPTION OF THE FIGURES

FIG. 1 depicts the effect of various lipids on the activation of mTOR signaling.

FIG. 2 depicts the increases in mTOR signaling with RIMFROST Sublime Krill Oil extract is much more modest compared to S-PS and S-PA but still significantly increased compared to baseline values, whereas S-PC elicited no increase. The samples were then subjected to Western blot analysis for p70-S6K phosphorylated on the threonine 389 residue (p′70-389) and total p70. The ratio of these signals was calculated and used as a marker of mTOR signaling. Values in the graphs represent the mean+SEM and were obtained from 2-3 independent experiments (n=4-12/group). * Significantly different from control (P<0.001). RIMFROST Sublime Krill Oil Extract significantly activates mTOR signaling.

FIG. 3 depicts increased mTOR signaling with increasing concentration of RIMFROST Sublime Krill Oil extract.

### DETAILED DESCRIPTION

The invention provides methods for activating the mTOR pathway, and effecting physiological processes regulated by the mTOR pathway.

mTOR Pathway

A protein kinase called the mechanistic target of rapamycin (mTOR) has been implicated in many aspects of cellular physiology or physiological processes, to include for example cellular growth and proliferation. In particular, mTOR has been implicated stimulation of protein synthesis to drive muscle hypertrophy. Accordingly, mTOR is also involved in muscle disuse atrophy, and age-related muscle loss. In fact, several studies have indicated that the kinase activity of mTOR is required for mechanically-induced increases in skeletal muscle protein synthesis and hypertrophy. Specifically, elevations in energy status, amino acids, and growth factors can increase MPS through, among other things, an mTOR-dependent mechanism. Furthermore, several studies have also shown that signaling by mTOR is required for mechanically induced increases in MPS and the ultimate hypertrophic response. (See, for example, Joy et al., Nutrition & Metabolism 2014, 11:29). Furthermore, protein synthesis is required for neurological function. Accordingly, activation of mTOR may reduce age-related cognitive decline and/or improve cognitive function.

### Krill Extract and Krill Extract Components

In one embodiment, the invention provides methods for activating the mTOR pathway, and effecting physiological processes regulated by the mTOR pathway by administering a composition including phospholipids, omega-3 fatty acids, and an antioxidant, or krill extract to a mammal or contacting said composition with a cell.

Examples of phospholipids suitable for use according to the present invention include phosphatidylcholine (PC) and alkylacylphosphatidylcholine (AAPC). Examples of omega-3 fatty acids suitable for use according to the present invention include eicosapentaenoic acid (EPA) and docosahexaenoic acid (DHA). Antioxidants suitable for use according to the present invention include alpha-tocopherol, flavonoid, or astaxanthin.

The phospholipids, omega-3 fatty acids, and antioxidants may be of krill origin.

The compositions containing phospholipids, omega-3 fatty acids, and an antioxidant, or a hill extract disclosed herein may further contain at least one of linoleic acid, alpha-linoleic acid, arachidonic acid, oleic acid, palmitic acid, palmitoleic acid, stearic acid, cholesterol, triglycerides, monoglycerides, all-trans retinol, canthaxanthin, (3-carotene, zinc, selenium, nervonic acid, sodium, potassium, and calcium.

Krill is the common name for small, shrimp-like crustaceans that swarm in dense shoals, especially in Antarctic waters. It is one of the most important food sources (especially protein) for fish, some kind of birds and especially for baleen whales. Nutritional value can be derived from hill by, for example, from the extract, protein components, phospholipid components, fatty acids, e.g., omega-3, and antioxidant components.

Krill extract is best characterized by its three major components: the phospholipids, the omega-3 fatty acids which are attached to the glycerol backbone of the phospholipids as well as the antioxidants, mainly astaxanthin which is responsible for the red color of the krill extract. The krill extract may be in the form of an oil. These three compositional features make hill extract different from any other omega-3 source. The major phospholipid in krill extract is phosphatidylcholine (PC) which is a molecule where the glycerol backbone has attached the nonpolar (hydrophobic) two fatty acids as well as the polar (hydrophilic) head group having a phosphate and choline unit which is the difference to nonpolar fatty acids in triglyceride form. Therefore phospholipids have a very unique structure, the bi-layer, which allows them to be the key building block of biological membranes.

The two major omega-3 fatty acids in krill are eicosapentaenoic acid (EPA) and docosahexaenoic acid (DHA) which represent the majority of the long-chain omega-3 fatty acids. These health promoting components are attached to the glycerol backbone of the phospholipids and are responsible for the hydrophobic character.

The major antioxidant in krill extract is astaxanthin which is a red-orange carotenoid pigment, a powerful biological antioxidant that occurs naturally in a wide variety of living organisms and acts as protector of human cells, protecting them from damage with potential onset of various diseases.

The composition of krill extract is best described by three different quality parameters with the following content (the method of quantification is provided in parenthesis).

TABLE 1
Typical composition of krill extract.

| | |
|---|---|
| Total Omega-3 Fatty Acids | >22% (AOCS Method Ce 1i-07) |
| Total Phospholipids | >40% (³¹P-NMR Method) |
| Astaxanthin | >150 ppm (HPLC Method) |

TABLE 2
Typical Phospholipid Composition of Krill Extract [weight %]

| Phospholipid (PL) | Weight Percentage [%] |
|---|---|
| Phosphatidylcholine (PC) | 76 |
| Alkyl acyl phosphatidylcholine (AAPC) | 7 |
| Phosphatidylinositol (PI) | 0.7 |
| Phosphatidylserine (PS) | 0.6 |
| Lysophosphatidylcholine (lyso-PC) | 6.5 |
| Lyso alkyl acyl phosphatidylcholine | 0.9 |
| Phosphatidylethanolamine (PE) | 3.2 |
| Alkyl acyl phosphatidylethanolamine (AAPE) | 1.9 |
| Lysophosphatidylethanolamine | 0.7 |
| Lyso alkyl acyl phosphatidylethanolamine | 0.2 |
| Other Phospholipids | 2.3 |

TABLE 3
Sample embodiment of the phospholipid content of krill extract.

| Phospholipid (PL) | Weight Percentage [%] |
|---|---|
| Phosphatidylcholine (PC) | 60-90 |
| Alkyl acyl phosphatidylcholine (AAPC) | 5-20 |
| Phosphatidylinositol (PI) | 0.1-20 |
| Phosphatidylserine (PS) | 0.1-20 |
| Lysophosphatidylcholine (lyso-PC) | 0.1-20 |
| Lyso alkyl acyl phosphatidylcholine | 0.1-10 |
| Phosphatidylethanolamine (PE) | 0.1-20 |
| Alkyl acyl phosphatidylethanolamine (AAPE) | 0.1-20 |
| Lysophosphatidylethanolamine | 0.1-10 |
| Lyso alkyl acyl phosphatidylethanolamine | 0.1-10 |
| Other Phospholipids | 0.1-20 |

In one embodiment, the compositions disclosed herein include less than 10%, less than 1%, less than 0.75%, less than 0.5%, less than 0.25%, less than 0.1%, less than 0.01% or less than 0.001% PS.

In one embodiment, the compositions disclosed herein include less than 10%, less than 1%, less than 0.75%, less than 0.5%, less than 0.25%, less than 0.1%, less than 0.01%, or less than 0.001% PA.

In one embodiment, the compositions disclosed herein include less than 10%, less than 1%, less than 0.75%, less than 0.5%, less than 0.25%, less than 0.1%, less than 0.01%, or less than 0.001% lysophosphatidic acid (LPA).

As used herein, a “hill extract component” includes one or more components enumerated in tables 1 and 2 that have been derived from krill.

In some embodiments, the krill extract is enriched for at least one component of krill. This composition is termed component enriched hill. As used herein, “enriched” refers to a composition fraction or portion wherein an object species has been partially purified such that, on a weight basis, the concentration of the Object species is higher than level of the species as disclosed in Table 1 (lower value for each species) or Table 2. For example, phospholipid enriched krill extract is a krill extract having at least 45% phospholipid. By way of additional example, phospholipid enriched krill extract is a krill extract having at least 50% phospholipid. By way of additional example, phospholipid enriched krill extract is a krill extract having at least 60% phospholipid.

In one embodiment, the hill extract is phospholipid enriched hill extract. In another embodiment, the hill extract is enriched for at least one of eicosapentaenoic acid (EPA), docosahexaenoic acid (DHA), phosphatidylcholine (PC), phsophatidylinositol (PI), phosphatidyl ethanolamine (PE), and sphingomyelin.

As used herein, when the composition of the present disclosure is enriched for more than one component, the sum of the components is enriched.

In one embodiment, the hill extract includes EPA and DHA.

In an embodiment, the hill extract includes at least 1%, at least 5%, at least 10%, or at least 15% EPA. In an embodiment, the krill extract includes at most 15%, at most 20%, at most 25%, or at most 30% EPA.

In an embodiment, the hill extract includes at least 1%, at least 5%, at least 10%, or at least 15% DHA. In an embodiment, the krill extract includes at most 15%, at most 20%, at most 25%, or at most 30% DHA.

In one embodiment, the hill extract includes 10-15% EPA and 5-15% DHA.

In one embodiment, the hill extract disclosed herein refers to RIMFROST Sublime Krill Oil Extract, which is of the nature and composition as described in Table 1, Table 2, and Table 3.

The krill extract or hill extract component disclosed herein may be obtained from any method known in the art. For example, hill extract may be obtained by solvent extraction. Examples of suitable solvents include ethanol, acetone, and hexane. In one embodiment, krill extract is obtained by supercritical solvent extraction. Other methods include, such methods as described in Yamaguchi et al., Supercritical carbon dioxide extraction of oils from Antarctic krill, J. Agric. Food Chem., 1986, 34 (5), pp 904-907, the contents of which are incorporated herein by reference.

The compositions described above may further contain non-toxic auxiliary agents, components, or ingredients that do not materially alter the basic and novel characteristics of the claimed methods.

Examples of non-toxic auxiliary agents, components or ingredients include pharmaceutically acceptable carriers. Suitable carriers and their formulations are described in Remington: The Science and Practice of Pharmacy (19th ed.) ed. A. R. Gennaro, Mack Publishing Company, Easton, Pa. 1995. By way of example, “Pharmaceutically acceptable carrier” includes one or more compatible solid or liquid fillers or excipients, which are suitable for human body and have sufficient high purity and low toxicity. “Compatible” herein means that each component of the composition can be blended with the compound of the present invention or with each other without remarkably reducing the pharmacodynamic activity of the compound. Some examples of pharmaceutically acceptable carrier includes sugars (e.g. glucose, sucrose, lactose, etc.), starch (e.g. maize starch, potato starch, etc.), cellulose and its derivatives (e.g. sodium carboxymethy cellulose, sodium ethyl cellulose, cellulose acetate, microcrystalline cellulose, etc.), polyethylene glycol, glutin, talc powder, stearic acid, magnesium stearate, calcium sulphate, vegetable oil (e.g. soybean oil, sesame oil, peanut oil, olive oil, etc.). It also may be emulsifier (e.g. Tween®), moistening agent (e.g. sodium dodecyl sulphate), coloring agent, flavoring agent, stabilizer, preservative, nonpyrogenic water, etc. The selection of carriers for the compound of the present invention depends on the administration mode of the compound. Preservatives include, for example, antimicrobials, anti-oxidants, and chelating agents.

Further carriers include sustained release preparations such as semipermeable matrices of solid hydrophobic polymers containing the delivery system, which matrices are in the form of shaped articles, e.g., films. It will be apparent to those persons skilled in the art that certain carriers may be more preferable depending upon, for instance, the route of administration and concentration of composition being administered.

The compositions described above may be formulated for topical administration and may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. The use of thickeners, flavorings, diluents, emulsifiers, dispersing aids or binders have also been contemplated and may be desirable.

In one embodiment, the only active compound for activating the mTOR pathway includes phospholipids, omega-3 fatty acids, and an antioxidant, or krill extract, and contains no other pharmaceutically active compound for activating the mTOR pathway.

In one embodiment, the only pharmaceutically active compound for of any kind includes phospholipids, omega-3 fatty acids, and an antioxidant, or krill extract, and contains no other pharmaceutically active compound.

The inventors of the invention unexpectedly discovered that the compositions disclosed herein activate the mTOR pathway. Without wishing to be bound by theory, it is believed that the unique properties of krill extract is imparted by the unique composition of krill, including the components and combinations of components.

In one embodiment, the invention provides a method of activating the mTOR pathway in a cell or in a mammal. In an exemplary embodiment, the method includes contacting a cell with a composition disclosed herein. The cell may be in vivo, ex vivo, or in vitro. Determination of activation may be made by assaying for the level of expression of a gene or protein within the mTOR signaling pathway. For example, P70S6 kinase (p70S6K) is in a signaling pathway that includes mTOR. mTOR can be activated in distinct ways, thereby activating p70S6K. The phosphorylation of P70S6K at threonine 389 has been used as a hallmark of activation by mTOR. Accordingly, activation of the mTOR signaling pathway may be determined by assaying for p70S6K phosphorylation.

In one embodiment, P70S6K phosphorylation is increased by at least about 10%, at least about 20%, at least about 50%, at least about 75%, at least about 100% as compared to p70S6K phosphorylation in the absence of the composition including phospholipids, omega-3 fatty acids, and an antioxidant, or a krill extract. Phosphorylation may be assessed by any method known in the art, including Western Blotting.

In a further embodiment, the present disclosure contemplates the use of one or more mTOR activators which may be co-administered with the composition of the present invention to give an additive or synergistic effect to the treatment or dosage regime. Such mTOR activators include protein, branch chained amino acids, leucine, leucine derivatives such as HMB (beta-hydroxyl-beta-methylbutyrate), HICA (hydroxy-isocaproic acid), ursolic acid, Phosphatidylserine (PS), Phosphatidic Acid (PA). Such activators may be administered separately, sequentially, or simultaneously with the composition described above.

Total muscle mass is the balance of muscle protein synthesis (MPS) and muscle protein breakdown (MPB). In yet another embodiment, the composition disclosed herein may be co-administered with agents that reduce muscle protein breakdown. Examples of such agents include HMB and HICA.

In one embodiment, the invention provides a method of augmenting muscle growth and/or strength effected by activation of mTOR pathway in a mammal in need thereof by administering the composition disclosed herein. In some embodiments, the composition described above may be administered to the mammal before, during, or after physical exercise.

As used herein, the term “augmenting” includes increasing, either alone or in combination with other effectors. Other effectors may include physical exercise.

As used herein, “physical exercise” includes aerobic exercise or resistance training based physical exercise. Physical exercise includes training in an exercise room, gym, or pool. Physical exercise does not include unsupervised, unprescribed routine movements such as casual walking or house work.

As used herein, “aerobic exercise” includes physical activity wherein the subject maintains an elevated heart rate (HR) for more than 15 minutes, more than 30 minutes, or more than 1 hour. Examples of aerobic exercise include jogging, walking, nordic walking, swimming, and cycling.

As used herein, “resistance training” includes the use of resistance to induce muscular contraction. Examples of resistance training include bench press, leg press, pulldown, leg curl, scull crushes, dumbbell lateral raise, dumbbell bicep curls, and barbell bicep curls.

Physical exercise includes a single session or a physical fitness regimen. In one embodiment, the physical fitness regimen includes at least one or at least two physical exercise sessions per day. In another embodiment, a physical exercise session is at least 15 minutes, at least 30 minutes, or at least 60 minutes.

The physical fitness regimen includes regular physical exercise. For example, the physical fitness regimen includes daily, every other day, or weekly exercise. As a further example, the physical fitness regimen includes 1-3 workouts per week, 2-4 workouts per week, or 3-5 workouts per week. The regimen may last for at least 1 month, at least 2 months, at least 3 months, at least 6 months, or at least 1 year.

In one embodiment, the physical fitness regimen includes aerobic exercise only, resistance based exercise only, or a combination of aerobic and resistance based physical exercise.

In one embodiment, augmenting muscle growth includes increasing muscle mass.

Muscle mass may be assessed by any method known in the art. For example, muscle mass may be assessed using dual energy x-ray absorbtiometery (DXA).

In one embodiment, muscle mass is increased by at least 1%, at least 2.5%, at least 5%, or at least 10% as compared to the muscle mass of the mammal prior to initiating the methods disclosed herein.

In one embodiment, augmenting strength includes increasing muscle strength. Strength may be assessed by muscle performance. Muscle performance can be assessed by the mammals performance on leg press, leg curls, standing calf raises, leg extensions, inclined leg lift, inverted situps (back extension), 45° inclined situps, bench press, latissimus pulldown, triceps pulldown, inclined dumbbell curls, seated preacher curls, seated rows, and CyBec Pec Fly.

In one embodiment, muscle strength is increased by at least 1%, at least 2.5%, at least 5%, or at least 10% as compared to the muscle strength of the mammal prior to initiating the methods described herein.

In another embodiment, in any of the methods disclosed herein, the composition described above is administered to a mammal before exercise. For example, the composition disclosed herein is administered at least 15, at least 30, at least 60, or at least 90 minutes before exercise. In another embodiment, in any of the methods disclosed herein, the composition or krill extract is administered to a mammal during exercise. In yet another embodiment, in any of the methods disclosed herein, the composition or krill extract is administered to a mammal after exercise. For example, the composition or hill extract is administered at least 15, at least 30, at least 60, or at least 90 minutes after exercise.

In another embodiment, in any of the methods disclosed herein, the composition described herein is administered to a mammal along with a meal. The meal may be breakfast, lunch, or dinner.

In one embodiment, in any of the methods disclosed herein, the composition disclosed herein is administered in a therapeutically effective amount. As used herein, a “therapeutically effective amount” means that the amount of the composition disclosed herein contained in the composition administered is of sufficient quantity to achieve the intended purpose, for example, activating the mTOR pathway, and effecting physiological processes affected by the mTOR pathway.

In another embodiment, in any of the methods disclosed herein, the composition disclosed herein is administered in a dose of 1-10 g per day. In another embodiment, the dose is 3 g of krill extract per day.

In embodiments wherein the method includes physical exercise, a first portion of the daily dose may be administered prior to exercise and a second portion of the daily dose may be administered after exercise. In one example, the first portion includes two thirds of the daily dose and is administered prior to exercise, and the second portion includes one third of the daily dose and is administered after exercise. On exercise free days, the mammal consumes the total amount dose with breakfast.

In one embodiment, the mammal is provided a total dose of 3 g of hill extract per day. On training days, the mammal consumes 2 g pre-workout, and 1 g post workout. On training free days, the mammal consumes the total amount (3 g) with breakfast.

In one embodiment, the entire daily dose is administered in the morning. In another embodiment, the entire daily dose id administered in the evening.

As used herein “dosage regimen” includes any combination of amount of the composition disclosed herein, at any interval disclosed herein, in conjunction with or without physical exercise.

In yet another embodiment, the invention provides a method of mediating muscle disuse atrophy effected by activation of mTOR pathway in a mammal in need thereof by administering a composition disclosed herein to the mammal before, during, or after physical exercise.

As used herein, the term “mediating” includes treating, reducing, or preventing.

As used herein, muscle disuses atrophy may be the result of a period of muscle disuse. The term “period of muscle disuse” as used herein, unless otherwise specified, refers to a period of muscle inactivity, including extended muscle inactivity, or full or partial immobilization of a body muscle resulting from bed rest, hospitalization, casting, and the like. In one specific embodiment, “period of muscle disuse” includes muscles in the arms or legs that have suffered from disuse, including extended disuse.

In one embodiment, the composition disclosed herein is administered to a mammal before, during, or after period of muscle disuse.

In one embodiment, once the period of muscle disuse is over, a composition disclosed herein may be administered during the period of muscle recovery for a period of at least one week, including at least one month, including at least six months, and including one year or longer facilitating muscle recovery. In one specific embodiment, a composition disclosed herein are administered for a continuous period of from one week to six months, including one month to six months following the period of muscle disuse. As noted above, the composition disclosed herein may also be administered during a portion or all of the period of muscle disuse.

In a further embodiment, the present disclosure contemplates the use of one or more muscle growth factors which may be co-administered with the composition of the disclosed herein to give an additive or synergistic effect to the treatment regime or methods disclosed herein. Such growth factors include HGF, FGF, IGF, MGF, growth hormone etc. Such substances may be administered separately, sequentially, or simultaneously with the composition disclosed herein.

In one embodiment, the invention provides a method of mediating age-related muscle loss (sarcopenia) effected by activation of mTOR pathway in a mammal in need thereof by administering the composition disclosed herein to the mammal before, during, or after physical exercise. Muscle loss may be assessed by assessing muscle mass.

In one embodiment, muscle mass is maintained over a period of time as compared to mammals who are not administered the composition disclosed herein. The period of time may be more than 1 year, more than 2 years, or more than 5 years.

In another embodiment, muscle mass may decrease at a lower rate than mammals who are not administered the composition disclosed herein.

In one embodiment, the invention provides a method of mediating age-related cognitive decline effected by activation of mTOR pathway in a mammal in need thereof by administering a composition disclosed herein to the mammal.

In one embodiment, the composition disclosed herein is administered to a mammal in conjunction with physical exercise or a physical fitness regimen.

The cognitive function in a mammal can be increased relative to a mammal of similar age that has not been administered a dosage form or regimen containing the composition disclosed herein. In some embodiments of the present invention, the increase in cognitive function is greater than 5%, or about 5% to about 90%, about 10% to about 80%, about 25% to about 75%, or about 30% to about 65% as measured by one of the assessment tests disclosed herein.

In some embodiments, administration of the composition disclosed herein increases cognitive function in a relatively short duration, e.g., 1 week to 26 weeks (week 1 to week 26). In some embodiments, the cognitive function in a mammal is increased by greater than 5%, about 5% to about 90%, about 25% to about 75%, or about 30% to about 65% on week 26. In some embodiments, the composition disclosed herein is administered daily for 1 week to 6 weeks (week 1 to week 6). In some embodiments, the cognitive function in a mammal is increased by greater than 5%, about 5% to about 90%, about 25% to about 75%, or about 30% to about 65% on week 6. In some embodiments, a composition disclosed herein is administered daily for 2 weeks to 4 weeks (week 2 to week 6). In some embodiments, the cognitive function in a mammal is increased by greater than 5%, about 5% to about 90%, about 25% to about 75%, or about 30% to about 65% on week 6. In some embodiments, a composition disclosed herein is administered daily for 28 days (day 28). In some embodiments, cognitive function in a mammal is increased by greater than 5%, about 5% to about 90%, about 25% to about 75%, or about 30% to about 65% by day 28.

Cognitive function can be assessed by any method known in the art. Examples of cognitive assessments are described in Table 4.

TABLE 4

| VARIABLE | DESCRIPTION | SOURCE |
|---|---|---|
| Matrix Reasoning | Inspect a matrix of geometric patterns | Raven (1962) |
| | and select the best completion of the | |
| | missing cell from a set of alternatives. | |
| Shipley Abstraction | Determine the best continuation of a | Zachary (1986) |
| | series of elements | |
| Letter Sets | Identify the set of letters that does not | Ekstrom, et al. (1976) |
| | follow the same rule as the other sets | |
| Spatial Relations | Determine which three-dimensional | Bennett, et al. (1997) |
| | figure corresponds to a two-dimensional | |
| | figure if it was assembled | |
| Paper Folding | Select the pattern of holes that would | Ekstrom, et al. (1976) |
| | result if a piece of paper was folded and | |
| | a hole punched in the specified location | |
| Form Boards | Select the pieces that could be | Ekstrom, et al. (1976) |
| | combined to fill a designated form | |
| Word Recall | Listen to a list of 12 unrelated words | Wechsler (1997) |
| | and recall as many as possible | |
| | immediately after the list | |
| Logical Memory | Listen to a brief story and immediately | Wechsler (1997) |
| | recall as many details as possible. | |
| Paired Associates | Listen to six pairs of unrelated words, | Salthouse, et al. (1996) |
| | and then recall the second member of | |
| | the pair when presented with the first | |
| | member. | |
| Digit Symbol | Refer to a code table to write the | Wechsler (1997) |
| | symbols paired with digits as rapidly as | |
| | possible. | |
| Pattern Comparison | Categorize pairs of line patterns as | Salthouse & Babcock (1991) |
| | “same” or “different” as rapidly as | |
| | possible. | |
| Letter Comparison | Categorize pairs of letter strings as | Salthouse & Babcock (1991) |
| | “same” or “different” as rapidly as | |
| | possible. | |
| Stroop Effect | Identify color of words and color that is | Stroop (1935) |
| | spelled by the words. | |
| Serial subtraction | Test ability to subtract a number from | Parker et al. (2010) |
| test (SST) | another number in serial | |

In one embodiment, cognitive function is assessed by a Stroop Test. The Stroop Test uses two trials. In the first test, the written color name differs from the color ink it is printed in, and the participant must say the written word. In the second test, the participant must name the ink color instead. The respondent does each task as quickly as possible within a time limit. This test measures selective attention, cognitive flexibility and processing speed, and it is used as a tool in the evaluation of executive functions. In particular, this test assesses the way that the brain automatically processes information in the presence of more mentally effortful tasks highlights the phenomenon of the interference effect. Making an appropriate response, when given two conflicting signals, challenges the brain's directional attention capacity, which is a foundational mental resource that allows us to voluntarily manage the focus of our thoughts and remain productive in the presence of other distracting stimuli in the environment around us.

Cognitive function may also be assessed by name-face-association test, the first-name/last-name test, remembering names (either immediately or one minute after introduction), and dialing a phone number by memory.

In some embodiments, mediating age-related cognitive decline is determined by comparing the cognitive function of the mammal being administered to a composition disclosed herein to a mammal of approximately the same age and physical condition, i.e., a peer. In some embodiments, the increase in cognitive function is determined by comparing the cognitive function of the individual before and after being administered a composition disclosed herein for 1 month, 2 months, 3 months, 6 months, 1 year, or 5 years, according to the invention for mammal being administered a composition disclosed herein.

One of skill in the art will appreciate that the amount of the increase can be dependent on various parameters, such as the initial cognitive function of the mammal. For example, in mammals having a reduced cognitive function, the amount of the increase in cognitive function can be greater, relative to a mammal with average cognitive function. The increase in cognitive function can also be dependent on the length and/or amount of administration of a composition disclosed herein, or the regimen of administration of a composition disclosed herein.

In animal model systems, cognitive function may be measured by any method known in the art, including using the following assessment tests: Morris water maze, Barnes circular maze, elevated radial arm maze, T maze or any other mazes in which subjects use spatial information. Other tests known in the art may be used to assess cognitive function, such as fear conditioning, novel object recognition, active avoidance, passive avoidance, illuminated open-field, dark activity meter, elevated plus-maze, two-compartment exploratory test or forced swimming test. In addition, cognitive function may be measured using imaging techniques such as Positron Emission Tomography (PET), functional magnetic resonance imaging (fMRI), Single Photon Emission Computed Tomography (SPECT), or any other imaging technique that allows one to measure brain function.

According to an embodiment of the invention, a method for treatment of age-related cognitive decline is provided in which composition disclosed herein is administered to a mammal aged 45 or older. According to an embodiment of the invention, a method for treatment of age-related cognitive decline is provided in which composition disclosed herein is administered to a mammal aged 50 or older. According to an embodiment of the invention, a method for treatment of age-related cognitive decline is provided in which composition disclosed herein is administered to a mammal aged 55 or older. According to an embodiment of the invention, a method for treatment of age-related cognitive decline is provided in which composition disclosed herein is administered to a mammal aged 60 or older. According to an embodiment of the invention, a method for treatment of age-related cognitive decline is provided in which composition disclosed herein is administered to a mammal aged 65 or older. According to an embodiment of the invention, a method for treatment of age-related cognitive decline is provided in which composition disclosed herein is administered to a mammal aged 70 or older. According to an embodiment of the invention, a method for treatment of age-related cognitive decline is provided in which composition disclosed herein is administered to a mammal aged 75 or older.

In one embodiment, the invention provides a method of improving long-term memory effected by activation of mTOR pathway in a mammal in need thereof by administering a composition disclosed herein to the mammal.

In some embodiments, mammals experience more than 5%, more than 10%, more than 15% improvement in long-term memory as compared to those who do not undertake the dosage regimens disclosed herein. Long-term memory can be assessed by any method known in the art. Examples of long-term memory assessments are described in Table 4.

Long-term memory is divided between procedural memories (knowing how) and declarative memories (know that). The declarative memories are divided semantic memories (general knowledge) and episodic memories (personal recollection). In this regard, testing semantic memory may provide an assessment regarding long-term memory. In particular, the category fluency test may be used to test semantic memory. The category fluency task is a simple test that measures the subject's capacity to generate words belonging to a specific category, for example the category of animals. When tested, the subject is given the instruction “I will now ask you to say out loud as many words as possible from a given category. Please tell me as many different kinds of animals as you can remember. You have one minute. The time starts now”.

In one embodiment, the invention provides a method of reducing fat mass in a mammal by administering a composition disclosed herein to the mammal.

In one embodiment, fat mass is decreased by at least 1%, at least 2.5%, at least 5%, at least 10%, or at least 15% as compared to the fat mass of the mammal prior to undertaking the methods disclosed herein.

Fat mass may be assessed by any method known in the art. For example, fat mass may be assessed using dual x-ray absorbtiometery (DXA).

The methods disclosed herein do not apply to the generalized administration of a composition disclosed herein, but rather the methods disclosed herein are directed to the administration of a composition disclosed herein for the purposes of effecting physiological processes affected by the mTOR pathway. In particular, the methods of the invention include augmenting muscle growth and/or strength, mediating muscle disuse atrophy, mediating age-related muscle loss, mediating age-related cognitive decline, increasing muscle mass, and reducing fat mass.

Throughout this specification, quantities are defined by ranges, and by lower and upper boundaries of ranges. Each lower boundary can be combined with each upper boundary to define a range. The lower and upper boundaries should each be taken as a separate element.

Reference throughout this specification to “one embodiment,” “an embodiment,” “one example,” or “an example” means that a particular feature, structure or characteristic described in connection with the embodiment or example is included in at least one embodiment of the present embodiments. Thus, appearances of the phrases “in one embodiment,” “in an embodiment,” “one example,” or “an example” in various places throughout this specification are not necessarily all referring to the same embodiment or example. Furthermore, the particular features, structures or characteristics may be combined in any suitable combinations and/or sub-combinations in one or more embodiments or examples. In addition, it is appreciated that the figures provided herewith are for explanation purposes to persons ordinarily skilled in the art and that the drawings are not necessarily drawn to scale.

As used herein, the terms “comprises,” “comprising,” “includes,” “including,” “has,” “having,” or any other variation thereof, are intended to cover a non-exclusive inclusion. For example, a process, article, or apparatus that comprises a list of elements is not necessarily limited to only those elements but may include other elements not expressly listed or inherent to such process, article, or apparatus.

“Consisting Essentially of” means that the methods and compositions may include additional steps, components, ingredients or the like, but only if the additional steps, components, or ingredients do not materially alter the basic and novel characteristics of the claimed methods and compositions. As used herein, components or ingredients that do not materially alter the basic and novel characteristics of the claimed methods and compositions include non-toxic auxiliary agents that do not detract from the benefits provided by the present formulations. These agents can, for example, facilitate the delivery and/or stabilize the composition with respect to its shelf life or its actual applications.

Further, unless expressly stated to the contrary, “or” refers to an inclusive “or” and not to an exclusive “or”. For example, a condition A or B is satisfied by any one of the following: A is true (or present) and B is false (or not present), A is false (or not present) and B is true (or present), and both A and B are true (or present).

Additionally, any examples or illustrations given herein are not to be regarded in any way as restrictions on, limits to, or express definitions of any term or terms with which they are utilized. Instead, these examples or illustrations are to be regarded as being described with respect to one particular embodiment and as being illustrative only. Those of ordinary skill in the art will appreciate that any term or terms with which these examples or illustrations are utilized will encompass other embodiments which may or may not be given therewith or elsewhere in the specification and all such embodiments are intended to be included within the scope of that term or terms. Language designating such nonlimiting examples and illustrations includes, but is not limited to: “for example,” “for instance,” “e.g.,” and “in one embodiment.”

In this specification, groups of various parameters containing multiple members are described. Within a group of parameters, each member may be combined with any one or more of the other members to make additional sub-groups. For example, if the members of a group are a, b, c, d, and e, additional sub-groups specifically contemplated include any one, two, three, or four of the members, e.g., a and c; a, d, and e; b, c, d, and e; etc.

A “subject” as used herein is any mammal, e.g., a human. Human subjects may also be referred to herein as patients. Patients are generally understood to be individuals under medical care, and are generally in need of treatment for a pathology disclosed herein. An “animal” or mammalian subject is any mammal customarily used in experimental model systems for assessing brain or cognitive function, such as a mouse, rat, or non-human primate. For purposes of this invention, mammalian subjects should be selected from a population, such as an outbred population, in which individuals can be characterized as aged impaired (AI), aged unimpaired (AU), and young (Y). The invention also encompasses animal subjects under veterinary care, e.g., companion animals, commercially valuable animals, animals of threatened or endangered species.

“Aged” is used herein to refer to mammals (e.g., rats) at or near the end of their average life span. Typically an aged rat would be about 24-30 months of age. An aged human would be seventy or more years of age.

“Young” refers to mammals (e.g., rats) at about the age of sexual maturity and when the hippocampus has just fully matured. Typically a young rat would be 6-9 months of age.

As used herein, unless otherwise noted, percentage values are weight percentage values.

In the specification, numerous specific details are set forth in order to provide a thorough understanding of the present embodiments. It will be apparent, however, to one having ordinary skill in the art that the specific detail need not be employed to practice the present embodiments. In other instances, well-known materials or methods have not been described in detail in order to avoid obscuring the present embodiments.

### EXAMPLES

The present invention is illustrated in further details by the following non-limiting examples.

### Materials and Methods

C2C12 myoblasts were plated at approximately 30% confluence and grown for 24 hours in 10% FBS High Glucose DMEM with antibiotics (100m/ml streptomycin and 100 U/ml penicillin; Sigma). At 16 hours prior to the experiment, myoblasts cells were switched to serum free high glucose DMEM (no antibiotics) and were approximately 70% confluent at the time of the experiment. All stimulants were dissolved in chloroform to yield a concentration of 10 mg/mL, with the exception of DAG which was dissolved at 2 mg/mL and G3P which was dissolved at 6 mg/mL. Each stimulant was then dried with a stream of nitrogen gas and resuspended in PBS to obtain either 20 or 60 nmol/100 μL, such that 100 μL added to 2 mL of media resulted in 10 or 30 μM respectively. Accordingly, cells were stimulated for 20 minutes with vehicle (Control; 100 μL of PBS) 10 or 30 μM of RIMFROST Sublime Krill Oil Extract (Rimfrost USA, Inc, Merry Hill, N.C.), soy-derived (S) phosphatidylserine (S-PS), phosphatidylinositol (S-PI), phosphatidylethanolamine (S-PE), phosphatidylcholine (S-PC), phosphatidic acid (S-PA), lysophosphatidic acid (S-LPA), diacylglycerol (DAG), glycerol-3-phosphate (G3P), or egg-derived PA (E-PA). Cells were then harvested in lysis buffer (40 mM Tris, pH 7.5; 1 mM EDTA; 5 mM EGTA; 0.5% Triton X-100; 25 mM (3-glycerophosphate; 25 mM NaF; 1 mM Na₃VO₄; 10 μg/mL leupeptin; and 1 mM PMSF) and subjected to immunoblotting as previously described. The ratio of P-p70-389 to total p70 was used as readout for mTOR signaling. As used in the Examples disclosed herein, 10 μM of RIMFROST Sublime Krill Oil Extract contains 0.01253 μM PS, which is 0.6% of the phospholipid content; and 30 μM of RIMFROST Sublime Krill Oil Extract contains 0.0376 μM PS, which is 0.6% of the phospholipid content.

The data was analyzed using a one way analysis of variance (ANOVA). A Tukey's Multiple Comparison Test was used to determine significant differences between treatments. Significance was set at P<0.05. Statistical analyses were performed on SigmaStat software (San Jose, Calif., USA).

### Example 1, Effect of Various Lipids on the Activation of mTOR Signaling

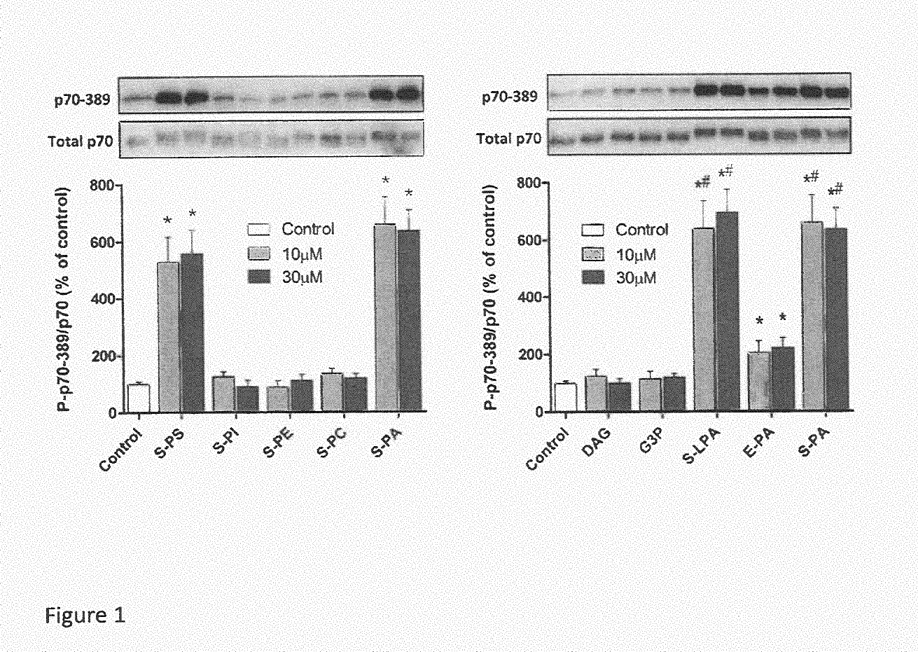

In accordance with the materials and methods described above, S-PS, S-PI, S-PE, S-PC, and S-PA were tested for their ability to activate mTOR signaling. See FIG. 1.

As can be seen, S-PI, S-PE, S-PC, DAG, and G3P elicited no increase in the ratio of P-p70-389 to total p70 compared to vehicle stimulated cells. In contrast, elevated mTOR signaling was observed at all tested concentrations of S-PS (529, and 558%), E-PA (206, and 221%), S-LPA (638, and 694%), and S-PA (658, and 636%; P<0.05). In addition, S-LPA and S-PA increased mTOR signaling to a greater degree than did E-PA at all concentrations (P<0.05).

Neither soy-derived PE nor PC were able to activate mTOR. Soy-derived phospholipids and phospholipids from Krill Oil differ in their fatty acid composition. Krill Oil is rich in omega-3 fatty acids, which are not present in soy-derived phospholipid. Comparison of soy-derived PA with egg-derived PA, which is rich in omega-3 fatty acids, showed that the omega-3 fatty acids to not beneficially improve mTOR activation.

### Example 2: Activation of the mTOR Pathway by Krill Extract and Phospholipids

In accordance with the materials and methods described above, krill extract, S-PC, S-PS, and S-PA were tested for their ability to activate mTOR signaling. The increases in mTOR signaling with RIMFROST Sublime Krill Oil extract is significantly increased compared to baseline values, whereas S-PC elicited no increase. The samples were then subjected to Western blot analysis for p70S6K phosphorylation on the threonine 389 residue (p′70-389) and total p70. The ratio of these signals was calculated and used as a marker of mTOR signaling. Values in the graphs represent the mean+SEM and were obtained from 2-3 independent experiments (n=4-12/group).* Significantly different from control (P<0.001).

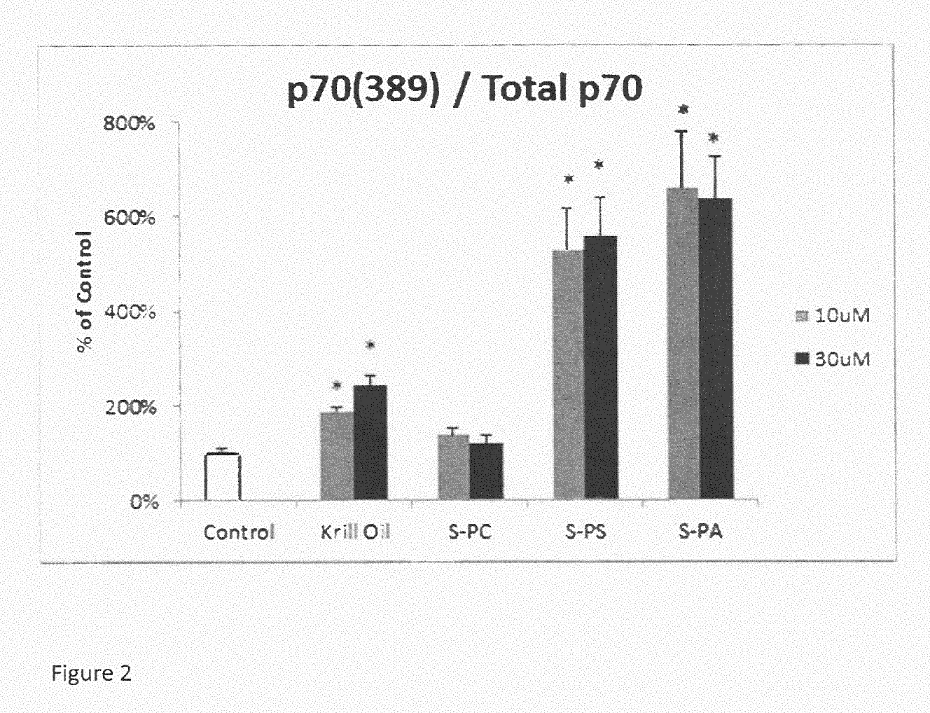

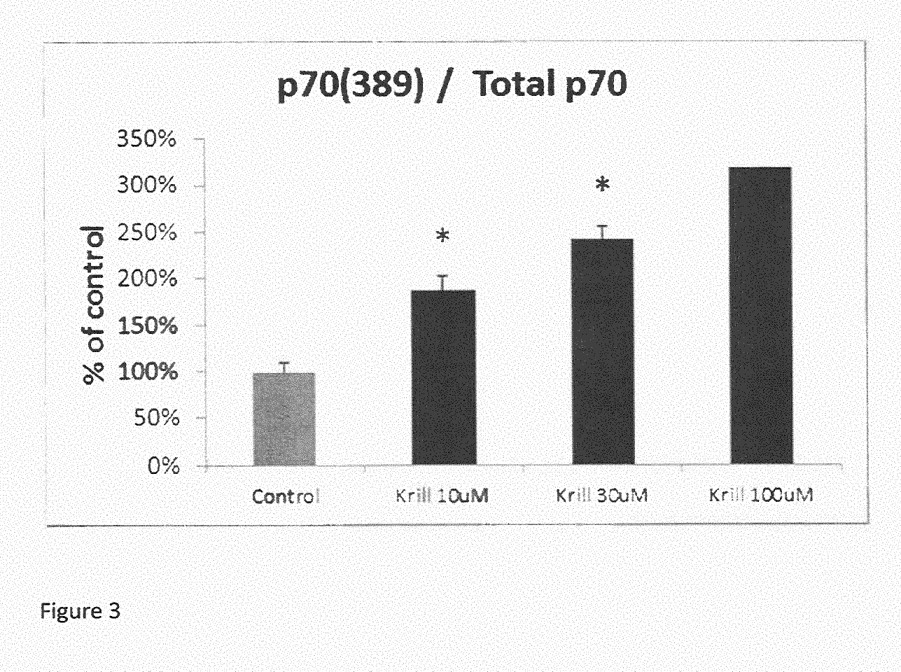

As shown in FIGS. 2 and 3, RIMFROST Sublime Krill Oil Extract significantly elevated mTOR signaling as compared to the control. Increasing the concentration of RIMFROST Sublime Krill Oil Extract increases mTOR signalling.

### Example 3: Activation of mTOR is Tested with Varying Concentration of Krill Extract Components

In accordance with the methods described above, hill extract components and equivalent phospholipids from other sources are individually tested to determine their ability to activate the mTOR pathway or increase mTOR signaling. Phospholipid components of hill including PC, AAPC, PI, PS, lyso-PC, PE, and AAPE from krill sources and non-hill sources.

### Example 4: The Effects of Krill Extract Supplementation Combined with Resistance Training on Body Composition and Athletic Performance

Male subjects, ranging in age of approximately 20-25, having at least 6 months of resistance training experience and have a minimum of 1 year of training experience are used to test the effects of krill extract supplementation.

Subjects are provided a total dose of 3 g of hill extract per day. On training days, the subjects consumed 2 g pre-workout, and 1 g post workout. On training free days, subjects consume the total amount (3 g) with breakfast.

As suggested by Kraemer et al. (2009), nutrition assessment screening are performed by a Registered Sports Dietitian to ensure that subjects are 1) on a diet consisting of 15-20% protein, 45-55% carbohydrate, and 25-30% fat; 2) not taking performance enhancing supplements for the last 6 weeks; 3) not smokers; 4) not taking amino acid supplements; 5) not using anabolic or catabolic hormones; and 6) not on medication or supplements known to influence any of the variables measured in the study. Subjects are carefully matched by age, body mass, strength and resistance training, and physical activity background, and randomly placed into one of the two groups.

Resistance exercise training protocol is a programmed, non-linear training split (4 days per week), as non-linear resistance-training program yield greater results than a traditional or non periodized program in athletes (Montiero et al., 2009). The program is designed to train all major muscle groups using a majority of compound movements for the upper body (e.g. bench press, dips, shoulder press, pullups, and bent over rows), lower body (leg press, leg extensions, GHR) and core. The programmed, non-linear training split is divided into hypertrophy days consisting of 8-10 RM loads for 3 sets, with 90 seconds rest, strength endurance days consisting of 12-15 repetitions, with 60 seconds rest, and heavy days consisting of 3 to 5 RM loads with 3 sets for all exercises except the leg press and bench press which will receive 5 total sets. Weights are progressively increased by 2-5% when the prescribed repetitions can be completed. All training sessions are closely monitored to ensure effort and intensity is maximal each training session. Subjects trained each body part twice weekly and alternate between hypertrophy, and heavy workouts. This protocol is selected as training at this frequency is ideal for moderately resistance trained individuals.

Strength Assessment: Maximal Strength assessment of bench press and squat is done prior to the initiation of the study (baseline), and weekly for 8-20 weeks.

Body Composition: Lean body mass, fat mass, and bone mineral density (both full body and regionally) are assessed using dual x-ray absorbtiometery (DXA).

### Example 5: The Effects of Krill Extract Supplementation on Muscle Mass

A double-blind study is performed over a 16-week period in subjects undergoing a physical exercise regimen. A baseline measurement of muscle mass and strength is taken prior to initiation of the study. Subjects are assigned to either a krill extract or placebo group. The dosage regimen includes 1-10 g of hill extract or 1-10 g of placebo per day.

The composition of the present disclosure is administered two to four times daily to a subject. Subjects were provided a total dose of 2-10 g of krill extract per day.

On training days, the subjects consumed 1-5 g pre-workout and 1-5 g post workout. On training free days, subjects consumed the total amount (10 g) with breakfast. After 10 weeks, increased muscle mass is observed than would be observed for those subjects not administered the composition of the present disclosure.

### Example 6: The Effects of Krill Extract Supplementation on Muscle Disuse Atrophy

A double-blind study is performed over a 16-week period in subjects having one limb immobilized. A baseline measurement of muscle mass and strength is taken prior to initiation of the study. Subjects are assigned to either a hill extract or placebo group. The dosage regimen includes 1-10 g of krill extract or 1-10 g of placebo per day.

The reduction of muscle mass of the immobilized limb is compared to the other non-immobilized limb and to the immobilized limb prior to initiation of the study.

Speed of recovery after cast is removed is also assessed. At the end of 8 weeks, the subjects initiate an exercise rehabilitation program. Measurements of strength and muscle mass are taken on a weekly basis. The placebo group is further divided into a hill extract and control group for the rehabilitation phase of the study.

The speed of recovery of subjects taking the krill extract, throughout the immobilization period is compared with those subjects taking the hill extract after the immobilization period.

### Example 7 the Effects of Krill Extract Supplementation on Age-Related Muscle Loss (Sarcopenia)

The effect of Krill supplementation on sarcopenia is measured by analyzing fractional synthetic rate (FSR) of myofibrillar protein in elderly men and women.

Men and women (50-79 years of age) with a body mass index (BMI) between 18 and 30 are recruited to participate in the study. Exclusion criteria includes diabetes (type 1 and 2), kidney disease (chronic renal disease), subjects with GI tract diseases, with a predisposition to hypertrophic scarring, arthritis, smokers, and subjects with milk allergies. In addition, body weight and height are assessed and body composition is determined by dual-energy X-ray absorptiometry (DXA). If deemed eligible, participants are randomized and counterbalanced into 4 treatment conditions (n=10 per condition) to ingest beverages containing either: 1.) 20 g of whey protein concentrate (WPC), 2.) 5 gwhey protein concentrate (WPC) plus 1-5 g of krill extract, 3.) 10 g whey protein concentrate (WPC) plus 1-5 g of krill extract, or 4.) water (as control). At least 1 week before the experimental infusion trial, participants undergo maximum strength tests to determine their unilateral twelve repetition maximum on a standard guided motion leg extension machine.

### Experimental Infusion

Participants arrive to the lab after an overnight fast. Subsequently, a Teflon catheter will be inserted in a heated dorsal hand vein for blood sampling. A second Teflon catheter is inserted into a vein in the opposite arm and participants will receive a priming dose of L-[ring-13C6]phenylalanine (2 mmol·kg−1) prior to initiating the continuous IV infusion of L-[ring-13C6]phenylalanine (0.05 mmol·kg−1·min−1). After the onset of the primed constant infusion (˜2.5 h), participants will perform an acute bout of unilateral resistance exercise consisting of 3 sets×12-15 repetitions on a guided motion leg extension machine. The exercise bout will be performed using a pre-determined load based on the each participant's 12 repetition maximum. A rest period of 2 min between each set will be provided. Immediately after exercise, biopsies from the vastus lateralis is collected from the exercise and non-exercise legs using a 5 mm Bergström needle modified for manual suction under local anesthesia. Subsequently, participants consume a randomly assigned nutrient treatment dissolved in 200 ml water. First, the nutrient powder is dissolved in the 150 ml of water, and any remaining powder is dissolved with 50 ml of water. And if any powder is still remaining in the cup, add the additional water to take all powder. Additional bilateral muscle biopsies are collected at 120 and 240 min from the exercise and non-exercise legs. Blood collections are taken every 0.5 or 1 h throughout the postprandial period.

Sample Analysis.

Plasma insulin concentrations are measured by a commercially available immunoassay kit (ALPCO Diagnostics). Blood glucose concentrations are measured on YSI 2300 STAT plus device (YSI Inc. Life Sciences, Yellow Springs, USA). Plasma amino acid concentrations and tracer enrichments are determined by conversion of the free amino acids to their TBDMS derivatives and GC-MS analysis. The skeletal muscle myofibrillar (contractile) proteins are extracted via differential centrifugation and protein bound enrichments are determined by LC/MS/MS analysis. Total protein content and phosphorylation status of mTORC1 at Ser2448 are determined by Western Blot analysis. Western blot data are normalized to an internal control (a-tubulin). The fractional synthetic rate (FSR) of myofibrillar protein are calculated from the determination of the rate of L[ring-2H5]phenylalanine incorporation into myofibrillar protein and using the plasma-free or intracellular free phenylalanine enrichment as the precursors pools.

### Example 8: The Effects of Krill Extract Supplementation on Age-Related Cognitive Decline

A double-blind study is performed over a 16-week period of subjects between 60 and 70 years of age. A baseline measurement of cognitive function is assessed by the Stroop test. Subjects are assigned to either a krill extract or a placebo group enabling two groups of similar sex and age to be obtained. Each group is further divided into exercise and non-exercise group. The exercise group is assigned an exercise regimen throughout the course of the study.

During the course of the study, subjects take a dose of 1-5 g of krill extract or 1-5 g of placebo per day. Cognitive function is assessed by any test described on Table 4 on a weekly basis.

Stroop Test measures will be taken at both pre and post exercise, at baseline and after 8 weeks of supplementation and resistance exercise training.

### Example 9: The Effects of Krill Extract Supplementation on Cognitive Function

A double-blind study is performed over a 16-week period of 20 subjects. A baseline measurement of cognitive function is assessed by the Stroop test at the beginning of the study. Subjects are assigned to either a krill extract or a placebo group enabling two groups of similar sex and age to be obtained. Each group is further divided into exercise and non-exercise group. The exercise group is assigned an exercise regimen throughout the course of the study. During the course of the study, subjects take a dose of 1-5 g of hill extract or 1-5 g of placebo per day.

Stroop Test measures will are taken at both pre and post exercise, and after 16 weeks of supplementation and exercise.

### Example 10: Testing Memory of Subjects after Administration of Krill Extract in Aged Mice Using an Object Recognition Test

Three groups of mice, having 10 mice in each group, is used for this test. Test mice aged fifteen months old (aged mice) are fed a diet having a composition that includes phospholipids, omega-3 fatty acids, and an antioxidant, or krill extract 3 times a week for three weeks. The composition described above is mixed with the normal mouse feed.

Two groups of mice are used as control. One control group comprised 10 young mice, aged 8 weeks, treated with vehicle without the above-referenced composition. Another control group includes 10 aged mice that are treated with vehicle without the above-referenced composition. Three weeks after the last administration, the cognitive function of the mice is tested by the Object Recognition Test, a behavioral assay that measures visual memory, based on the natural tendency of mice to explore novel objects. In the first day of the experiments, the mice are familiarized with two objects in the experimental arena. The mice did not dissociate between the two objects and spent similar time in exploring each of them. Twenty-four hours later, the mice are introduced to the same arena with one of the familiarized objects replaced by a novel object. Control young mice (YOUNG) remember the old object and preferred to explore the novel object, as is expressed by statistically significant longer exploration time of the new object relative to the old object. Untreated aged mice (AGED) lose this ability and do not spend more time exploring the new object than the old object, indicating that they had failed to remember the old object. Aged mice that express similar behavior as the young mice, i.e., spend significantly more time exploring the new object relative to the old object is indicates that they remember the old object. Relative exploration time equals exploration time for a given object, divided by exploration time of both object.

### Example 11: Testing Age-Related Cognitive Decline in an Animal Model Using the Morris Water Maze Test

The effect of hill extract on age-related cognitive decline is investigated in an animal model by the Morris Water Maze Test. The Morris water maze test measures learning and memory in a place-navigation environment. A large circular pool filled with water is used as the test apparatus. The water is darkened with brown food coloring and maintained at a certain temperature. Four equidistant points at the edge of the pool are designated as start positions and a transparent platform is fixed 1 cm below the surface. The rats are trained in two blocks of four trials each, using all four starting positions in a random sequence. If the rat fails to reach the hidden platform within 120 seconds, it will be placed there by the researcher. Aged rats (21-24 months) will be investigated and performance is compared to young, five-month-old rats. Age-dependent decline is not uniform throughout the population since rats, like humans, develop cognitive impairments to a variable degree. Rats are screened in the Morris water maze test to determine and select aged impaired and aged non-impaired rats. Aged impaired rats will be matched by mean performance scores and then assigned to a hill extract or control group. After the initial screening test the rats will be tested again after 7 and 12 weeks of oral hill extract supplementation.

While there have been described what are presently believed to be the preferred embodiments of the present invention, those skilled in the art will realize that other and further changes and modifications may be made thereto without departing from the spirit of the invention, and it is intended to claim all such modifications and changes as come within the true scope of the invention.

1.-5. (canceled)

6. A method of increasing muscle mass effected by activation of mTOR pathway in a mammal comprising administering a composition comprising krill extract to said mammal before, during, or after physical exercise.

7. A method of reducing fat mass effected by activation of mTOR pathway in a mammal comprising administering a composition comprising krill extract to said mammal before, during, or after physical exercise.

8. A method activating the mTOR pathway in a cell comprising contacting said cell with an effective amount of a composition comprising krill extract.

9. A method according to claim 6, wherein said krill extract is enriched for phospholipids.

10. The method according to claim 7, wherein said krill extract is enriched for phospholipids.

11. The method according to claim 8, wherein said krill extract is enriched for phospholipids.

12.-15. (canceled)

16. A method according to claim 6, wherein administering comprises administering 1-10 g of krill extract per day.

17. A method according to claim 6, wherein administering comprises administering 1-5 g of krill extract per day.

18.-36. (canceled)

37. The method according to claim 6, wherein the composition is administered in conjunction with a physical fitness regimen.

38. The method according to claim 37, wherein the physical fitness regimen includes at least one of aerobic exercise and resistance training based exercise.

39. The method according to claim 38, wherein the physical exercise regimen comprises a aerobic or resistance based physical fitness regimen lasting at least four weeks.

40. A method according to claim 7, wherein administering comprises administering 1-10 g of krill extract per day.

41. A method according to claim 7, wherein administering comprises administering 1-5 g of krill extract per day.

42. The method according to claim 7, wherein the composition is administered in conjunction with a physical fitness regimen.

43. The method according to claim 42, wherein the physical fitness regimen includes at least one of aerobic exercise and resistance training based exercise.

44. The method according to claim 43, wherein the physical exercise regimen comprises a aerobic or resistance based physical fitness regimen lasting at least four weeks.

45. The method according to claim 8, wherein the cell comprises a muscle cell.
